(19) United States
(12) Patent Application Publication
(10) Pub. No.: US 2026/0066114 A1
(43) Pub. Date: Mar. 5, 2026

(54) MEDICAL ARTIFICIAL GENERAL INTELLIGENCE (MAGI)

(71) Applicant: GEORGE MASON UNIVERSITY, Fairfax, VA (US)

(72) Inventor: Farrokh ALEMI, McLean, VA (US)

(21) Appl. No.: 19/253,342

(22) Filed: Jun. 27, 2025

Related U.S. Application Data
(60) Provisional application No. 63/687,913, filed on Aug. 28, 2024.

(51) Int. Cl. G16H 50/20; G16H 10/60; G16H 20/00

(52) U.S. Cl. CPC G16H 50/20; G16H 10/60; G16H 20/00

(57) ABSTRACT

A Medical Artificial General Intelligence (MAGI) system can provide recommendations for medical diagnoses or treatments responsive to a patient's medical history. Information regarding the medical history can be obtained via an intake module, for example, utilizing a large language model, which may interact with a user in a conversational manner. Patient-specific features can be extracted from the medical history information and correlated with variables in a predetermined knowledgebase, which includes data indicative of an order of occurrence of the variables. The MAGI system can create a reduced knowledgebase responsive to the patient-specific features, which can enable the MAGI system to identify one or more candidate medical diagnoses or responses to medical treatments with computationally efficiency.

### CROSS-REFERENCE TO RELATED APPLICATION(S)

The present application claims the benefit of and priority under 35 U.S.C. § 119(e) to and is a non-provisional of U.S. Provisional Application No. 63/687,913, filed Aug. 28, 2025, and entitled “Artificial Autonomous Clinician,” which is hereby incorporated by reference herein in its entirety.

### STATEMENT REGARDING FEDERALLY SPONSORED RESEARCH

This invention was made with government support under OD032581 awarded by the National Institutes of Health. The government has certain rights in the invention.

### FIELD

The present disclosure relates generally to computerized systems and methods for suggesting medical treatments and/or diagnoses, and more particularly, to medical artificial general intelligence systems and methods, for example, capable of computationally efficient operation to provide hallucination-free suggestions.

### BACKGROUND

Artificial General Intelligence (AGI) systems have been developed to perform a variety of cognitive tasks traditionally associated with human intelligence, including the understanding and processing of natural language. These systems have demonstrated capabilities in reasoning and decision-making based on linguistic inputs. However, the application of AGI in the medical field presents challenges, particularly in providing accurate medical diagnoses and treatment recommendations. The demand for precise and contextually relevant information within complex medical data challenges the capabilities of existing AGI systems.

For example, current AGI systems often rely on linguistic relationships derived from published medical literature to infer treatment effectiveness. This approach may be limited by biases in published studies, where negative results are frequently underreported, potentially leading to inaccuracies in medical advice. Additionally, these systems may struggle to differentiate between linguistic word associations and causal medical concept relationships, which may be important to understand medical treatment effectiveness. AGI systems may also produce incorrect outputs, or “hallucinations,” when faced with insufficient data, as they attempt to provide answers based on available information. In general, such systems do not ask for clarity, do not check sufficiency of available information, do not un-learn non-causal associations, and do not actively engage in improving the data collection during the conversation phase. Furthermore, challenges arise in interpreting unreported data, usually assumed as not present, but may in fact be probable.

Embodiments of the disclosed subject matter may address one or more of the above-noted problems and disadvantages, among other things.

### SUMMARY

Embodiments of the disclosed subject matter provide systems and methods for Medical Artificial General Intelligence (MAGI), for example, to provide automated advice on medical diagnoses and/or treatments based on patient-specific inputs. In some embodiments, a MAGI system can leverage a comprehensive knowledgebase constructed from structured medical records and ontologies to handle a wide range of medical conditions and treatments. In some embodiments, a MAGI system can enhance the accuracy of medical advice by reducing misinformation and hallucinations commonly associated with conventional Artificial General Intelligence (AGI) systems. For example, in some embodiments, a MAGI system can employ causal analysis to eliminate spurious associations in observational data and can ensure that sufficient information is gathered from the patient before providing a diagnosis or treatment recommendation.

In some embodiments, a MAGI system can employ and/or interact with an intake module configured to interact with users, for example, to collect medical history information of a patient through conversation. In some embodiments, the intake module can employ a large language model, for example, to facilitate interaction with the user, to ensure that all necessary variables are accounted for, and/or to solicit any missing information from the user. In some embodiments, In some embodiments, a MAGI system can dynamically adjust its knowledgebase based on patient-reported information, ensuring that the advice provided is tailored to the patient's medical history while reducing computational load. Moreover, the MAGI system's ability to converse with the user allows it to verify the presence or absence of critical variables, thereby improving the accuracy of its recommendations and reducing the likelihood of confounding in the data.

In one or more embodiments, a method can comprise receiving, at an intake module, medical history information of a patient. The method can further comprise extracting at least one patient-specific feature from the received medical history information. Each feature can correspond to a medical history event, a medical diagnosis, a medical symptom, a medical procedure, a medication, a treatment, a response to treatment, or a laboratory finding. The method can also comprise correlating, via a medical analysis module, each feature to at least one of a plurality of variables in a predetermined knowledgebase. The predetermined knowledgebase can include data indicative of an order of occurrence for the plurality of variables, and the plurality of variables can include a plurality of candidate output variables.

The method can further comprise creating, via the medical analysis module, a reduced knowledgebase from the predetermined knowledgebase based at least in part on the correlated variables. The method can also comprise analyzing, via the medical analysis module, the reduced knowledgebase with respect to at least one of the plurality of candidate output variables. The method can further comprise identifying, via the medical analysis module, one or more of the candidate output variables based at least in part on the analyzing of the reduced knowledgebase. The plurality of candidate output variables can comprise different medical diagnoses or different responses to medical treatments.

In one or more embodiments, a system can comprise one or more processors, one or more databases, and one or more non-transitory media. The one or more databases can store a predetermined knowledgebase including data indicative of an order of occurrence for a plurality of variables. The variables can include a plurality of candidate output variables comprising different medical diagnoses or different responses to medical treatments. The one or more non-transitory media can store computer-readable instructions that, when executed by the one or more processors, cause the one or more processors to perform functions of one or more modules, including a medical analysis module.

The medical analysis module can be configured to receive at least one patient-specific feature corresponding to a medical history event, a medical diagnosis, a medical symptom, a medical procedure, a medication, a treatment, a response to treatment, or a laboratory finding. The medical analysis module can further be configured to correlate the at least one feature to at least one of the variables in the predetermined knowledgebase, and to create a reduced knowledgebase from the predetermined knowledgebase based at least in part on the correlated variables. The medical analysis module can also be configured to analyze the reduced knowledgebase with respect to at least one of the plurality of candidate output variables, and to identify one or more of the candidate output variables based at least in part on the analysis on the reduced knowledgebase.

Any of the various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosed technology will become more apparent from the following detailed description, which proceeds with reference to the accompanying figures.

### BRIEF DESCRIPTION OF THE DRAWINGS

Embodiments will hereinafter be described with reference to the accompanying drawings, which have not necessarily been drawn to scale. Where applicable, some elements may be simplified or otherwise not illustrated in order to assist in the illustration and description of underlying features. Throughout the figures, like reference numerals denote like elements.

FIGS. 1-2 are simplified schematic diagrams illustrating aspects of a Medical Artificial General Intelligence (MAGI) system, according to one or more embodiments of the disclosed subject matter.

FIG. 3 illustrates operational aspects of components of a MAGI system, according to one or more embodiments of the disclosed subject matter.

FIGS. 4A-4B are process flow diagrams for a MAGI method, according to one or more embodiments of the disclosed subject matter.

FIG. 5 depicts a generalized example of a computing environment in which the disclosed technologies may be implemented.

FIG. 6 illustrates an exemplary creation of a network model by the MAGI system based on patient-reported variables.

FIG. 7 illustrates aspects of an exemplary calculation of variable direct effects by the MAGI system.

### DETAILED DESCRIPTION

### General Considerations

For purposes of this description, certain aspects, advantages, and novel features of the embodiments of this disclosure are described herein. The disclosed methods and systems should not be construed as being limiting in any way. Instead, the present disclosure is directed toward all novel and nonobvious features and aspects of the various disclosed embodiments, alone and in various combinations and sub-combinations with one another. The methods and systems are not limited to any specific aspect or feature or combination thereof, nor do the disclosed embodiments require that any one or more specific advantages be present, or problems be solved. The technologies from any embodiment or example can be combined with the technologies described in any one or more of the other embodiments or examples. In view of the many possible embodiments to which the principles of the disclosed technology may be applied, it should be recognized that the illustrated embodiments are exemplary only and should not be taken as limiting the scope of the disclosed technology.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one skilled in the art.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person skilled in the art to have a more definitive construction, the numerical parameters set forth are approximations that may depend on the desired properties sought and/or limits of detection under standard test conditions/methods, as known to those skilled in the art. When directly and explicitly distinguishing embodiments from discussed prior art, the embodiment numbers are not approximates unless the word “about,” “substantially,” or “approximately” is recited. Whenever “substantially,” “approximately,” “about,” or similar language is explicitly used in combination with a specific value, variations up to and including 10% of that value are intended, unless explicitly stated otherwise.

Directions and other relative references may be used to facilitate discussion of the drawings and principles herein but are not intended to be limiting. For example, certain terms may be used such as “inner,” “outer,” “upper,” “lower,” “top,” “bottom,” “interior,” “exterior,” “left,” right,” “front,” “back,” “rear,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part, and the object remains the same.

As used herein, “comprising” means “including,” and the singular forms “a” or “an” or “the” include plural references unless the context clearly dictates otherwise. The term “or” refers to a single element of stated alternative elements or a combination of two or more elements unless the context clearly indicates otherwise.

Although there are alternatives for various components, parameters, operating conditions, etc. set forth herein, that does not mean that those alternatives are necessarily equivalent and/or perform equally well. Nor does it mean that the alternatives are listed in a preferred order, unless stated otherwise. Unless stated otherwise, any of the groups defined below can be substituted or unsubstituted.

Unless explained otherwise, all technical and scientific terms used herein have the same meaning as commonly understood to one skilled in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Features of the presently disclosed subject matter will be apparent from the following detailed description and the appended claims.

### Introduction

Disclosed herein are systems and methods for Medical Artificial General Intelligence (MAGI), for example, to provide automated advice on medical diagnoses and treatments based on patient-specific inputs. In some embodiments, the MAGI system can leverage a comprehensive knowledgebase constructed from structured medical records and ontologies and can address a wide range of medical conditions and treatments. Moreover, the MAGI system can enhance the accuracy of medical advice by mitigating misinformation and hallucinations commonly associated with conventional Artificial General Intelligence (AGI) systems. By employing causal analysis, the MAGI system can eliminate spurious associations in observational data and ensure that sufficient information is gathered about the patient before providing a diagnosis or treatment recommendation.

In some embodiments, the MAGI system can interact with an intake module configured to collect patient medical history information, for example, in a conversational manner via large language models. This interaction can ensure that all necessary variables are accounted for, as well as allow any missing information to be collected from the user. The MAGI system dynamically adjusts its knowledgebase based on patient-reported information, tailoring the advice to the patient's medical history while reducing computational load. The system's ability to converse with the user allows it to verify the presence or absence of critical variables, thereby improving the accuracy of its recommendations and reducing the likelihood of confounding in the data.

In some embodiments, suggestions or recommendations for medical treatments and/or diagnoses can be providing while avoiding, or at least reducing hallucinations and/or misinformation by using a source of knowledge not otherwise available to conventional AGI systems or medical professionals. In some embodiments, the MAGI system can rely on the structured medical record diagnoses, treatment, and outcome codes found in electronic health records to guide the conversation in Large Language Models (LLM) of the intake model. This knowledgebase is objective data and based on experiences of patients with different treatments and/or diagnoses. In contrast, conventional AGI systems rely on associations between words in published reports, which can be biased since negative results are generally not published. Since the knowledgebase employed in embodiments of the disclosed subject matter has access to both the negative and positive findings, hallucinations can be reduced and the reliability of the recommendations can be improved, as compared to conventional AGI systems.

Moreover, in some embodiments, cause-and-effect conditional probabilities can be determined based on relationships among medical history variables, unlike conventional AGI systems that focus on the relationship among words. For example, in some embodiments, causal analysis can be employed to robustly unlearn spurious associations in observational data, which may otherwise distort inferences about treatment effectiveness and/or medical diagnoses. Embodiments of the disclosed subject matter can thus remove the confounding in observational data before making inferences and can further reduce hallucinations and improve accuracy by ignoring spurious associations.

Embodiments of the disclosed subject matter can further determine if sufficient (e.g., necessary) information has been provided in order to make a treatment recommendation or medical diagnosis. In contrast, conventional AGI systems provide answers based on available information, even when this information is insufficient, which can increase the occurrence of hallucinations. In some embodiments, when insufficient information has been supplied, further information can be requested, for example, by entering into additional conversations with the user to gather the missing patient information. In some embodiments, the MAGI system may refrain from providing a diagnosis or treatment advice unless and until sufficient information is available.

In some embodiments, the MAGI system can be configured to manage the conversation between the intake module (e.g., the LLM) and the client. Such management can include, but is not limited to, instructing the intake module to ask the client to clarify a point they have made earlier, asking the client to discuss a topic that they have not mentioned, asking the client to move to a different topic, asking the client leading questions to rule out alternative explanations, and other steps for directing long conversations over multiple topics. As such, the MAGI system can improve accuracy of inferences by actively guiding the data collection effort in the intake module.

Models utilized in embodiments of the disclosed subject matter can be built based on the patient-specific information, for example, dynamically while interacting with the patient (or other user). Thus, embodiments of the disclosed subject matter can avoid, or at least reduce, any mismatch between patient-reported data and the data required by internal algorithms employed by the MAGI system. In contrast, conventional AGI systems build models on the concepts in its knowledgebase, appropriate for the entire population, and then instantiate it on patient's reported data, leading to a mismatch between patient reported medical history and the minimum, often optimally organized, input needed in AGI. Because of this mismatch, data needed for assessing treatment effectiveness may not be supplied by the patient to the AGI, and thus the performance of such conventional systems may rapidly deteriorate due to the missing information.

### Medical Artificial General Intelligence (MAGI) Systems

In some embodiments, software components, applications, routines or sub-routines, or sets of instructions for causing one or more processors to perform certain functions may be referred to as “modules” or “engines.” It should be noted that such modules or engines, or any software or computer program referred to herein, may be written in any computer language and may be a portion of a monolithic code base, or may be developed in more discrete code portions, such as is typical in object-oriented computer languages. In addition, the modules or engines, or any software or computer program referred to herein, may in some embodiments be distributed across a plurality of computer platforms, servers, terminals, and the like. For example, a given module or engine may be implemented such that the described functions are performed by separate processors and/or computing hardware platforms. Further, although certain functionality may be described as being performed by a particular module or engine, such description should not be taken in a limiting fashion. In other embodiments, functionality described herein as being performed by a particular module or engine may instead (or additionally) be performed by a different module, engine, program, sub-routine or computing device without departing from the spirit and scope of the invention(s) described herein.

It should be understood that any of the software modules, engines, or computer programs illustrated herein may be part of a single program or integrated into various programs for controlling one or more processors of a computing device or system. It should be understood that the client interacting with the MAGI system, or particular components thereof, may not be aware of which part of the MAGI system may be directing the operations, interactions, and/or activities of the MAGI system, or particular components thereof. Further, any of the software modules, engines, or computer programs illustrated herein may be stored in a compressed, uncompiled, and/or encrypted format and include instructions which, when performed by one or more processors, cause the one or more processors to operate in accordance with at least some of the methods described herein. Of course, additional and/or different software modules, engines, or computer programs may be included, and it should be understood that the examples illustrated and described with respect to the figures herein are not necessary in any embodiments. Use of the terms “module” or “software engine” is not intended to imply that the functionality described with reference thereto is embodied as a stand-alone or independently functioning program or application. While in some embodiments functionality described with respect to a particular module or engine may be independently functioning, in other embodiments such functionality is described with reference to a particular module or engine for ease or convenience of description only and such functionality may in fact be a part of, or integrated into, another module, engine, program, application, or set of instructions for directing a processor of a computing device.

In some embodiments, the instructions of any or all of the software modules, engines or programs described above may be read into a main memory from another computer-readable medium, such from a read-only memory (ROM) to random access memory (RAM). Execution of sequences of instructions in the software module(s) or program(s) can cause one or more processors to perform at least some of the processes or functionalities described herein. Alternatively or additionally, in some embodiments, hard-wired circuitry may be used in place of, or in combination with, software instructions for implementation of the processes or functionalities described herein. Thus, the embodiments described herein are not limited to any specific combination of hardware and software.

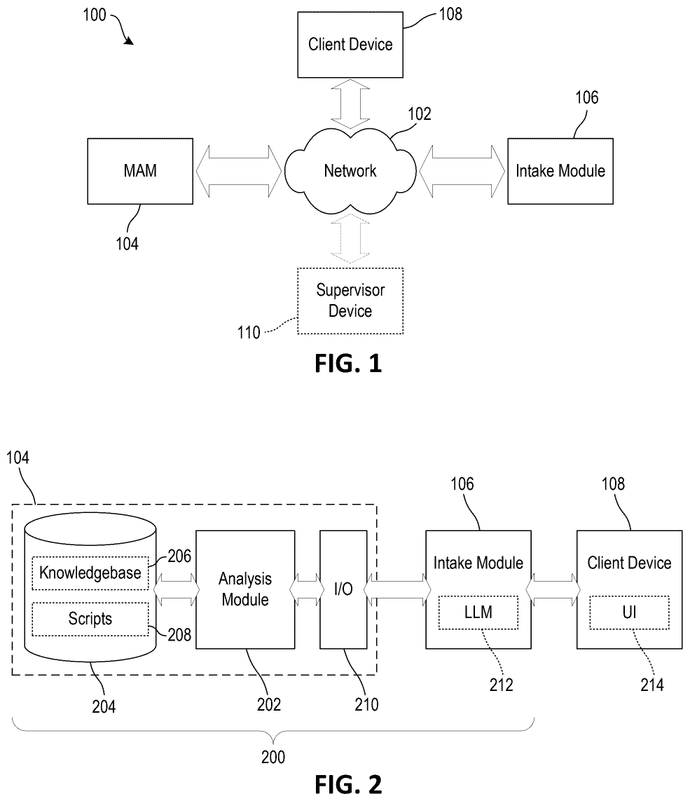

Referring now to FIG. 1, an exemplary configuration 100 for operation of a MAGI system is shown. In the illustrated example, a medical analysis module 104, an intake module 106, and one or more client devices 108 can communicate with each other via a network 102 (e.g., wired or wireless). Network 102 can include any combination of networking hardware and software used to establish communications the various computing systems. For example, the network 102 can be an Internet area network (IAN), a wide area network (WAN), a local area network (LAN) connected to a WAN, or any other network configuration or combinations thereof. In some embodiments, client device 108 (and/or optional supervisor device 110) may be part of an enterprise network separate from that of the intake module 106 and/or the medical analysis module 104. In such embodiments, network 102 can include hardware (e.g., modems, routers, switches, etc.) and software (e.g., firewall software, security software, billing software, etc.) to establish a networking link between the client device 108 (and/or supervisor device 110) and the Internet, and between the Internet and the intake module 106 and/or the medical analysis module 104. It should be appreciated that the network setup illustrated in FIG. 1 has been simplified and that many more networks and networking devices can be utilized to interconnect the various computing systems disclosed herein. Alternatively, in some embodiments, the medical analysis module 104 and intake module 106 can be co-located and/or part of a common system, for example, such that communication between medical analysis module 104 and intake module 106 is via an internal or private network (e.g., LAN). Other configurations are also possible according to one or more contemplated embodiments.

Via the client device 108 and network 102, a user (e.g., patient, patient representative, healthcare professional, etc.) can initiate a session with the MAGI system and provide medical history information of the patient to intake module 106, for example, in a conversational manner. In some embodiments, patient-specific features can be extracted from the medical history information (e.g., by the intake module 106) and provided to the medical analysis module 104 via the network 102. After analysis of its predetermined knowledgebase based on the patient-specific features, the medical analysis module 104 can output a recommendation (or recommendations) of a diagnosis or treatment to intake module 106 via network 102, for example, with instructions to communicate the recommendation to the user at client device 108 in a particular manner (e.g., hallucination-free manner). In some embodiments, one or more supervisor devices 110 can optionally be provided, for example, such that a human (e.g., healthcare professional, etc.) can review communications between client device 108 and the intake module 106 to monitor for signs or characteristics that require medical intervention (e.g., suicidal tendencies), to correct any hallucinations or mis-statements by the intake module 106, or for any other purpose.

Referring to FIG. 2, further details of a MAGI system 200 are shown. In the illustrated example, the MAGI system 200 includes medical analysis module 104 and intake module 106. Alternatively, in some embodiments, the MAGI system may include the medical analysis module 104 with or without the intake module 106 (or aspects thereof). The medical analysis module 104 can include an input/output module 210, an analysis module 202, and one or more databases 204. Alternatively, in some embodiments, the medical analysis module 104 may include analysis module 202 and input/output module 210 with or without the database(s) 204 (or aspects thereof). In the illustrated example, database(s) 204 can store a knowledgebase 206 and scripts 208. Alternatively, in some embodiments, separate database(s) can be provided for each of knowledgebase 206 and scripts 208. Alternatively or additionally, in some embodiments, the knowledgebase 206 and/or scripts 208 can be stored by a remote database from and accessible by (e.g., via network 102) the medical analysis module 104.

In some embodiments, the knowledgebase 206 in database 204 can be predetermined (e.g., built, organized, or otherwise established), for example, prior to any interaction with a user via client device 108. For example, the predetermined knowledgebase 206 can be organized around pairs of variables, where each variable can reflect a medical history event, a medical diagnosis, a medical symptom, a medical procedure, a medication, a treatment, a response to treatment, a laboratory finding, an outcome, or any other feature of patients or their medical history that may be helpful or instructive in making a treatment recommendation or diagnosis. These concepts are represented in electronic health records and ontologies as structured codes, accompanied with English language descriptions. In some embodiments, in addition to the variables already in electronic health records, the knowledgebase 206 can include one or more features constructed from these variables, for example, response to treatment and/or history of response to treatment. For example, in modeling effectiveness of antidepressants, the response to antidepressants may not be formally listed in the electronic health record. Rather, the response can be inferred from continuation of the medication, for example, for more than 10 weeks without augmentation or switching. Alternatively or additionally, history of response to antidepressants may not be directly provided in the structured variables in electronic health records and can be derived for inclusion in the knowledgebase 206.

Variables within the knowledgebase 206 can be represented in a binary fashion, for example, “1” if a particular variable has occurred or is present, or “0” if a particular variable is absent. Some variables may be related to each other; for example, diagnoses are often related to treatment/procedures. In a graphical representation of the variables in the knowledgebase 206, an arc can show the association between any two variables, with the direction of the arc indicating the temporal occurrence (going from the preceding event to the subsequent event). In some embodiments, conditional likelihoods or odds ratios can be calculated in the knowledgebase 206, for example, to provide an indication of the strength of association between any two variables. Conditional likelihoods differ from other measures of associations, such as correlations, since these measures are not symmetric, i.e., L(Xᵢ|Xⱼ)+L(Xⱼ|Xᵢ). For example, for variables Xᵢ and Xⱼ (e.g., two variables that are present in the population), the likelihood of Xᵢ occurring after the occurrence of Xⱼ, can be calculated as:

$$L ( {X}_{i} \vert {X}_{j} ) = \{ \begin{matrix}1 + C ( {X}_{i} ⋂ {X}_{j} ) & if C ( {X}_{i} ⋂ ¬ {X}_{j} ) = 0 \\ \frac{1}{1 + C ( {X}_{i} ⋂ ¬ {X}_{j} )} & if C ( {X}_{i} ⋂ {X}_{j} ) = 0 \\ \frac{C ( {X}_{i} ⋂ {X}_{j} )}{C ( {X}_{j} )} & Else\end{matrix}$$

where C( ) is the count of unique patients having the specified features. For example, C(Xᵢ∩¬Xⱼ) is the count of patients who have Xᵢ but not Xⱼ. In some embodiments, variable relationships whose conditional likelihoods that are less than a first threshold value (e.g., δ<0.15) may be omitted and/or ignored (e.g., as not informative), and/or variable relationships for C(Xⱼ) less than a second threshold value (e.g., C(Xⱼ)<30 or <10) may be omitted and/or ignored (e.g., as insufficient sample size).

In some embodiments, the knowledgebase 206 can further include information regarding and/or indicative of the order of occurrence of any pairs of variables. For example, information on a patient's diagnosis, medication prescription, or procedure is time-stamped inside existing medical records. In addition, order can be deduced from definition of some of the variables (e.g., death would occur as the last medical condition) and/or by the percent of variation explained by the conditional probability. In some embodiments, if a timestamp was used, then the order of occurrence for the variables can be determined based on the timestamps. For example, the number of times the first occurrence of Xᵢ precedes the first occurrence of variable Xⱼ can be counted, when both variables have occurred for the same person. In addition, if Xᵢ has occurred but Xⱼ has not yet occurred, it can be assumed that Xᵢ precedes Xⱼ because when, and if, it eventually occurs, it will meet the order requirement. The resulting formula for establishing the order between a pair of variables can be given by:

$$p ( {X}_{i} << {X}_{j} ) = \frac{C ( {X}_{i} << {X}_{j} ) + C ( {X}_{i} ⋂ ¬ {X}_{j} )}{N}$$

where:
p(Xᵢ<<Xⱼ) represents the probability that Xᵢ occurs before Xⱼ;
C(Xᵢ<<Xⱼ) is the count of unique patients in which first Xᵢ occurs before first Xⱼ;
C(Xᵢ∩¬Xᵢ) is the count of unique patients in which Xᵢ has occurred, but Xⱼ has not; and
N is the total number of unique patients.

In massive data, when the temporal order of a pair of variables is not available, this order can be estimated from the frequency information alone:

$$p ( {X}_{i} << {X}_{j} ) = \frac{C ( {X}_{i} ∩ ¬ {X}_{j} )}{N} .$$

In some cases, paired comparisons of order of variables can be intransitive and contradictory. For example, a circular triad may produce a temporal order where k occurs before j, which occurs before i, which illogically also occurs before k. This may occur when two variables occur in proximity to each other, such that the two variables may occur (or are otherwise documented in the medical records) in reverse order of their expected values, thereby creating contradictory order of occurrences. To understand if two variables occur too close to each other to make their order of occurrence unreliable, a Coefficient of Internal Reliability, θ, can be calculated prior to or as part of the storing in knowledgebase 206. For example, this Coefficient can be calculated from the frequency of intransitive triads as:

$$ϑ = 1 - \frac{C}{{C}_{max}} {C}_{max} = \frac{r ( {r}^{2} - 1 )}{24} for odd r , {C}_{max} = \frac{r ( {r}^{2} - 4 )}{24} for even r$$

where C is the count of circular triads related to the pair of variables and r is the number of variables examined.

In some embodiments, when initially organizing and/or subsequently refining knowledgebase 206 (e.g., retraining the MAGI system), circular triads can be reduced by re-defining variables so that they are further apart (e.g., on average, several months apart). Alternatively or additionally, a “double sorted” matrix can be used to identify the pair of variables that is most responsible for intransitive circular errors. Once the pair of variables responsible for most circular triads has been identified, the definition of one of the two variables can be revised, for example, by splitting into one of the two variables into new separate variables (e.g., “V prior to U”, and “V after U”). For example, if obesity and diabetes are responsible for significant circular triads, then obesity can be defined as two variables: “obesity prior to diabetes” and “obesity post diabetes.” The circular variables can be redefined in this fashion until the overall “Coefficient of Internal Reliability”, θ, is no longer statistically significant.

In some embodiments, knowledgebase 206 can also include the marginal probabilities of the variables. For example, the marginal probability can be calculated as:

$$p ( {X}_{i} ) = \frac{c ( {X}_{i} )}{N}$$

where c(Xᵢ) is the count of unique patients who have at least one occurrence of the variable Xᵢ, and the constant Nis the unique number of patients from which the knowledgebase was calculated.

In some embodiments, the construction of knowledgebase 206 (e.g., with (a) conditional likelihoods, (b) temporal orders of pairs of variables, and (c) marginal probabilities of variables) can offer significant advantages. For example, since the knowledgebase 206 does not include patient level data, it can be extracted from electronic health records without a violation of privacy (e.g., Health Insurance Portability and Accountability Act (HIPAA) rules). Moreover, if the knowledgebase 206 is accidentally leaked, it will not affect patients' privacy. Indeed, in some embodiments, the knowledgebase 206 may be in the public domain or otherwise available to the public.

In some embodiments, the information used to build the knowledgebase 206 can be extracted from multiple databases, without having to create federated databases to process the data. For example, multiple databases can be merged together by simply adding the counts of variables across these databases. Moreover, since the variables in the knowledgebase 206 employ counts, the size of knowledgebase 206 may be relatively small (e.g., as compared to the massive amount of patient data typically available in an electronic health record or the Medicare database). For example, given the terms employed for diseases, medications, and procedures (estimated to be less than 96,000 terms), the entire knowledgebase 206 can be about 18.4 gigabytes (e.g., using single precision 32-bit floats).

As noted above, a user (e.g., patient, healthcare personnel, or other human) can interact with the MAGI system 200 via client device 108 to request a medical treatment recommendation or medical diagnosis, for example, based on their medical history and/or symptoms. In some embodiments, the client device 108 can include a user interface 214 (e.g., display, keyboard, mouse, etc.) via which the user can provide input to and receive output from the intake module 106. Alternatively or additionally, intake module 106 and/or client device 108 can provide a web-based graphical user interface for interaction with the user. Information derived from the intake module 106 based on interactions with the user via client device 108 can be conveyed to the analysis module 202, for example, via input/output module 210, for subsequent processing to determine a medical treatment recommendation or medical diagnosis specific to the medical history reported by the user. The determined treatment recommendation or diagnosis can then be returned to the intake module 106, along with scripted instructions from the predetermined scripts 208, for reporting to the user via client device 108, for example, in a substantially hallucination-free manner.

In some embodiments, the input to the MAGI system 200 comes from patient interactions with Large Language Models (LLMs) 212, which can be effective in classifying concepts mentioned by the patient. LLMs are a large number of Neural Network models of the intake module 106 (or one or more separate models accessible by the intake module 106) that are used to generate responses to queries and carryout a conversation with the user. These models can be made ahead of time and can reflect the relationship among variables in the population (e.g., as defined in knowledgebase 206). In some embodiments, at the time of use, these population models can be instantiated to the features that are present in the patient, for example, where all variables that are in the population model but absent in the patient are assumed to have a value of zero. Thus, the conversation between the intake module 106 and the user can yield a reduced knowledgebase based on the user-specified features of the patient, which can enhance computational processing speed and/or recommendation accuracy, among other advantages.

### Medical Artificial General Intelligence (MAGI) Methods

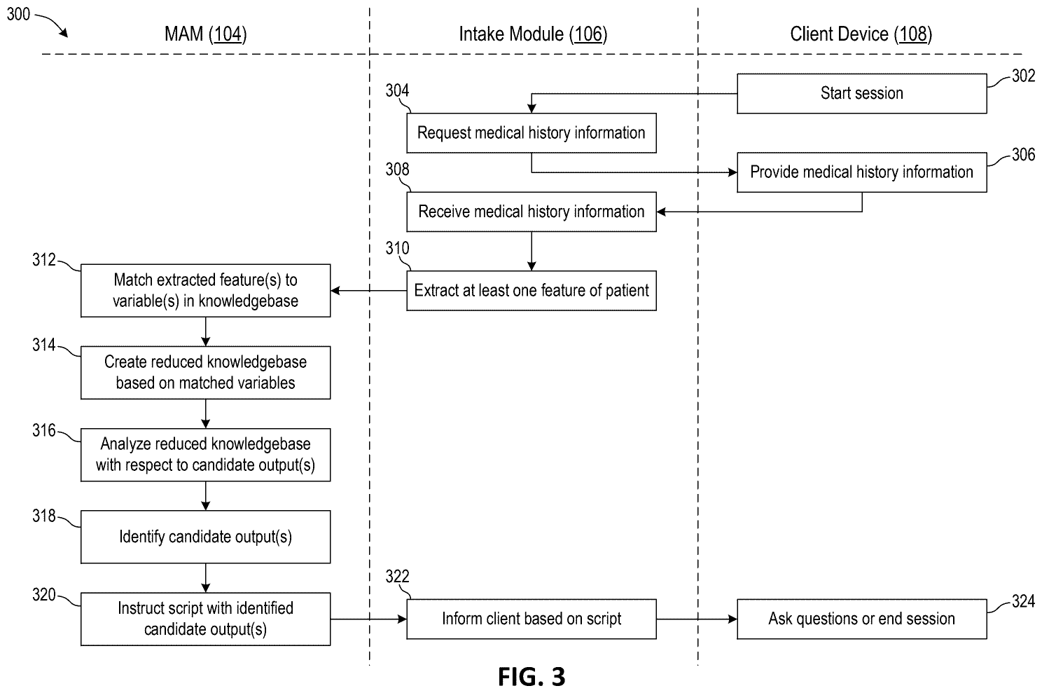

FIG. 3 illustrates exemplary interactions between a client device 108, intake module 106, and medical analysis module 104, as part of an operational flow 300 of a MAGI system. The process can begin at 302, where a user starts a session, for example, by sending a request via client device 108 or otherwise initiating contact with conversational module 106 (e.g., by accessing a MAGI website). In response to the start of the session, the intake module 106 can prompt the user (e.g., via text and/or audio prompts) at 304 for certain information (e.g., desired recommendation and/or medical history), and the user can respond at 306 with the requested information. In some embodiments, the interactions 304, 306 between the user and intake module 106 may be repeated, for example, such that information is requested and provided in a conversational manner. Alternatively or additionally, in some embodiments, at least some of the information may be provided (e.g., uploaded) as a data or a document (e.g., medical records from the user or a healthcare provider) by client device 108 or from a third-party device (e.g., separate data connection to a medical record database).

Based on the received information at 308, the intake module 106 can extract at least one patient-specific feature at 310. For example, each extracted feature can correspond to a medical history event, a medical diagnosis, a medical symptom, a medical procedure, a medication, a treatment, a response to treatment, an outcome, or a laboratory finding. The extracted features can then be sent to the medical analysis module 104, where, at 312, each extracted feature can be correlated (e.g., matched) to one or more variables in the predetermined knowledgebase. Alternatively or additionally, in some embodiments, the medical analysis module 104 can evaluate the information from the client device 108 with respect to one or more criteria, for example, to check for clarity and/or completion of the intake information. For example, if the information obtained by the intake module is determined to be incomplete and/or unclear, the intake module 106 can be directed to ask follow-up, probing, and/or clarifying questions, and the client device 108 can respond to such questions. Alternatively or additionally, the intake module 106 can be directed to rule-out alternative explanations and/or signal an end of a topic to the client device 108.

Based on the correlated variables, the medical analysis module 104 can create, at 314, a reduced knowledgebase specific to this patient's medical history. At 316, this reduced knowledgebase can then be analyzed by the medical analysis module 104 to identify one or more candidate output variables (e.g., a medical diagnosis and/or favorable response to medical treatment) in the knowledgebase that may be applicable to the patient. For example, the medical analysis module 104 can employ an algorithm to create a directed acyclical graph that has paths going through the patient-specific data. Such a graph is not based on any unreported variables (except for unreported events that have led to selection of treatment) in the knowledgebase; rather, any unreported variables are interpreted as not having occurred and thus irrelevant to the analysis. The larger network of relationships in the predetermined knowledgebase can thus be reduced to a computationally more efficient, smaller network based on patient specific data.

Once one or more candidate output variables have been identified at 318, the medical analysis module 104 can send, at 320, the output(s) to the intake module 106, along scripted instructions on how to report to the user. Using the script and based on the output(s), the intake module 106 can inform the user, at 322, of the recommended treatment and/or medical diagnosis, for example, in a hallucination-free manner. At 324, the user may be allowed the opportunity to ask follow-up questions or otherwise terminate the session.

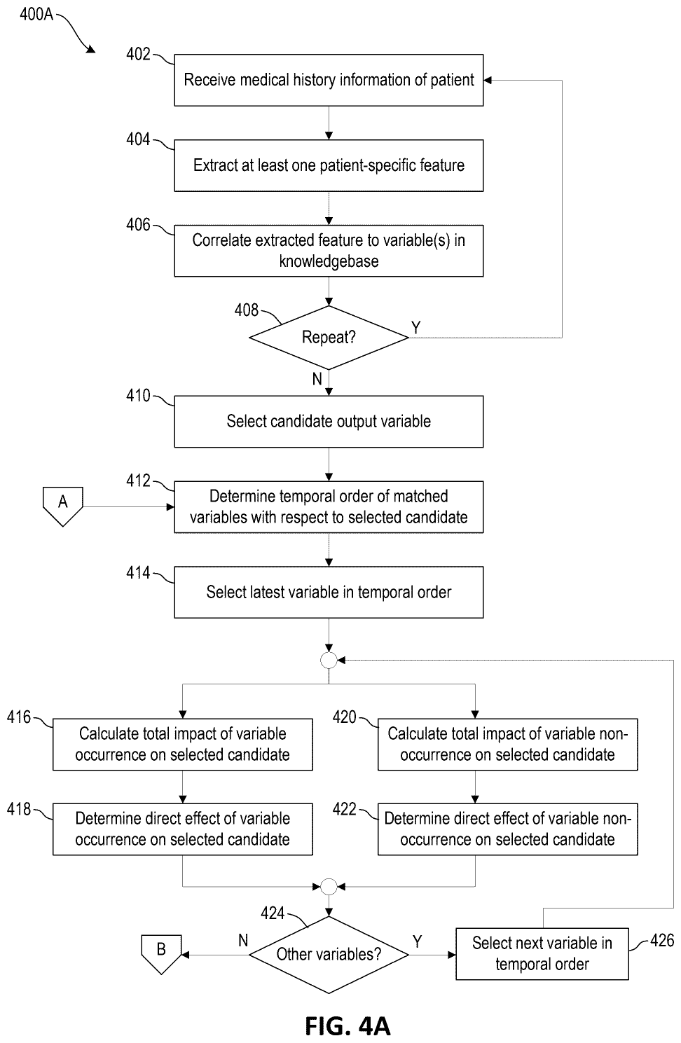

FIGS. 4A-4B illustrate further details of an operational method 400 of a MAGI system. Referring initially to FIG. 4A, method 400A can initiate at process block 402, where medical history information regarding a patient can be received. In some embodiments, the medical history information can be provided in an unstructured narrative or colloquial form, for example, in response to questions and/or as part of a conversation between an intake module (e.g., LLM) and the user. The method 400A can proceed to process block 404, where at least one patient-specific feature can be extracted from the received medical history information, and then to process block 406, where the extracted feature is correlated (e.g., matched) to one or more variables in the predetermined knowledgebase (e.g., structured codes from medical ontology).

For example, a user may report to the intake module that they have “sugar disease.” Based on this received medical history information, the intake module (or part of the medical analysis module) can extract (e.g., recognize) a patient-specific feature of “diabetes.” In the Systemized Nomenclature of Medicine (SNOMED) ontology of diseases, “diabetes” matches 258 structured codes, two of which are (1) Type II diabetes mellitus (code X40J5), and (2) Type I diabetes mellitus (code X40J4). In some embodiments, the matching of process block 406 can account for this mismatch between what is specified by the user and what variables are present in the knowledgebase. For example, the user may provide a term (e.g., Z) that is more general than the variables in the knowledgebase, and multiple variables (e.g., Xᵢ codes), which are more granular, may match the term. An adjusted conditional likelihood can be calculated as:

$$L ( {X}_{j} \vert Z ) = \frac{{\sum }_{z} C ( {X}_{z} ∩ {X}_{j} )}{{\sum }_{z} C ( {X}_{z} )} {X}_{z} ∈ Z$$

where C( ) indicates count of events, and Z is a general term that contains many granular terms X_{z}. The order between general terms Zᵢ and Zⱼ can be given as:

$$p ( {Z}_{i} << {Z}_{j} ) = \frac{1}{\vert {Z}_{i} \vert \vert Zj \vert} {\sum }_{X ∈ Z i} {\sum }_{Y ∈ Z j} P ( X << Y )$$

where p(Zᵢ<<Zⱼ) is the probability that the event Zᵢ occurs before event Zⱼ across all patients, |Zᵢ| is the number of events in the set Zᵢ, and |Zⱼ| is the number of events in the set Zⱼ. The double summation can iterate over all pairs of events i and j, averaging the precedence probabilities between the two sets. When p(Zᵢ<<Zⱼ) is larger than the reverse, then it can be assumed that Zᵢ preceded Zⱼ.

Alternatively or additionally, the user may provide a term that is more granular than the variables in the knowledgebase. The granular term, Z, in the user's expression can inherit the properties of the more general variable term, Xᵢ, in the knowledgebase, for example, as given by:

$$L ( {X}_{j} \vert Z ) = L ( {X}_{j} \vert {X}_{i} ) Z ϵ {X}_{i}$$

where Z is a more granular expression or term that is a part of or related to Xᵢ, and the conditional likelihood for the granular term Xⱼ given Z is equivalent to that of Xⱼ given Xᵢ. Similarly, the probability that Zᵢ preceding Xⱼ can be the same as the probability that Xᵢ preceding Xⱼ, given that Zᵢ is more granular version of Xᵢ, for example, as given by:

$$p ( {Z}_{i} << {X}_{j} ) = p ( {X}_{i} << {X}_{j} ) Z ϵ {X}_{i} .$$

Alternatively or additionally, the outcome of interest may be broader than the conditional likelihood in the knowledgebase, and the ontology of outcome variables can be used to find a more general term. Alternatively or additionally, the user may express a concept that does not match any available structured codes or variables in the knowledgebase, in which case, the intake module can be instructed to go back to the user for more information (e.g., to seek more clarity regarding what was meant by the user).

The method 400A can proceed to decision block 408, where it is determined if further information is needed from the user. In some embodiments, the decision on whether further information is needed may be determined by the intake module, for example, based on a predetermined minimum amount of information corresponding to a particular candidate output variable, or set of variables. For example, the intake module may proceed through a series of questions (e.g., age, gender, PHQ-9 questionnaire) to obtain a desired initial set of information for a particular medical condition (e.g., depression). Alternatively or additionally, the decision on whether further information is needed may be determined by the medical analysis module, for example, based on variables previously determined to be significant or important to a particular candidate output variable, or set of variables.

If it is determined at decision block 408 that additional information is initially needed, the method 400A may return to process block 402, for example, to ask for and receive additional information from the user via the intake module. Otherwise, the method 400A may proceed to process block 410, where a first candidate output variable in the knowledgebase is selected for evaluation, and then to process block 412, where a temporal order of patient-specific variables in the knowledgebase is determined with respect to the selected candidate output variable. For example, the knowledgebase can have the following inputs to determine order of occurrence:
(1) A set of n binary variables Z and a binary outcome Y. The Z variables are variables providing the features of the patient. In some cases, some of these variables may not be provided in the initial intake of process blocks 402-406, but rather added later (e.g., at process block 432 in response to decision block 430) in response to a determination by the algorithm regarding missing necessary variables.
(2) Pairwise conditional probabilities between any two variables can be calculated from contingency tables reporting presence or absence of the two variables, including calculating from contingency tables the following likelihoods:

$$L ( {Z}_{i} = 1 \vert {Z}_{j} = 1 ) , L ( {Z}_{i} = 1 \vert {Z}_{j} = 0 ) , L ( Y = 1 \vert {Z}_{j} = 1 ) , L ( Y = 1 \vert {Z}_{j} = 0 )$$

(3) Count of times one variable occurs before another, shown as C(Zᵢ<<Zⱼ), where << indicates the relationship of one variable temporally occurring before another, and C indicates a count of times when Zᵢ occurs before Zⱼ for the same person plus the count of times Zᵢ occurs but Zⱼ does not occur for the same person. In some cases, it can be assumed that if Zⱼ ever occurs it would be after Zᵢ.

For each variable Zₖ, the score for number of events that precede it can be calculated as:

$$Score ( {Z}_{k} ) = {\sum }_{j \neq k} C ( {Z}_{j} << {Z}_{k} ) - C ( ( {Z}_{k} << {Z}_{j} ) )$$

where a higher score indicates that more variables precede Zₖ. In some embodiments, the variables can then be sorted based on the score to produce an order of the variables, with the first occurring variable being 1 (least events before it) and the last independent variable being indexed as n (most independent variables occurring before it). In some embodiments, the candidate output value (e.g., Y) can be assumed to be a dependent variable and occurring after all independent variables. The order of variables can thus be given by:

$${Z}_{1} << {Z}_{2} << … << {Z}_{n} << Y .$$

When multiple variables are present, some of the variables may be correlated and the association among the variables may distort the predictions based on total effect of variables. Instead, in some embodiments, the analysis can rely on only the direct effect of variables. For example, the direct effect of each patient-specific variable can be calculated through a recursive procedure that starts from the last variable and moves backward until the first variable in the determined temporal order. The direct effect D_{k,Y}, can provide an indication of how much influence variable k has on candidate output variable Y that is not already captured by downstream variables. Note that this is not necessarily a causal relationship; rather, this analysis recognizes that variable k may increase the probability of Y.

The method 400A can thus proceed to process block 414, where the latest variable (e.g., immediately prior to the selected candidate output value) in the temporal order of variables can be selected. The method 400A can then determine the total impact of the occurrence of the selected variable on the candidate output variable at process block 416 and use the determined total impact to calculate the direct effect of occurrence of the selected variable on the candidate output variable at process block 418. The method 400A can also determine the total impact of non-occurrence of the selected variable on the candidate output variable at process block 420 and use the determined total impact to calculate the direct effect of non-occurrence of the selected variable on the candidate output variable at process block 422, which determinations may occur at the same time as process blocks 416-418 or at a different time (e.g., after direct effects of occurrence of all variables in the temporal order have been determined).

In some embodiments, instead of separately calculating impacts of occurrence and non-occurrence in process blocks 416 and 420 and determining respective direct effects in process blocks 418 and 422, the method can calculate the total odds of impact of a change in the selected variable on the candidate output variable, and then determine the direct odds of impact of the selected variable on the candidate output variable, for example, as described in detail in the MAGI Processing Examples below.

If other variables remain in the temporal order at decision block 424, the method 400A may proceed to decision block 426 where the next latest variable in the temporal order can be selected and process blocks 416-422 repeated to provide a recursive estimation of the direct effect of each patient-specific variable on the selected candidate output variable.

In some embodiments, the input to the recursive estimation process can include:
a list of variables in temporal order Z₁<<Z₂<< . . . <<Zₙ<<Y,
a contingency table from which the Total effect of variable k on Y, can be calculated as an odds ratio:

$${T}_{k Y} = \frac{L ( Y = 1 \vert {Z}_{k} = 1 )}{L ( Y = 0 \vert {Z}_{k} = 1 )} / \frac{L ( Y = 1 \vert {Z}_{k} = 0 )}{L ( Y = 0 \vert {Z}_{k} = 0 )} ,$$

and
the weight Lambda used to balance the direct and indirect effects by the proportion of times downstream variables k+i occur after variable k has occurred and measured as

$${λ}_{k , k + i} = P ( {Z}_{k + i} = 1 \vert {Z}_{κ} = 1 ) .$$

In some embodiments, the recursive algorithm can be initialized by setting D_{n,Y}=T_{n,Y}, since the last variable does not have any indirect effects, and direct and total effects are the same. In the initialization step, the index k can also be set to n. For the recursive step, k can be set to k−1, and the procedure can iterate until k equals 1. The total effect can be composed of probability weighted sums of direct and indirect effects. For example, the direct effect can be calculated as the portion of total effect not explained by downstream indirect total effects, as given by:

$${D}_{k , Y} = \frac{{T}_{k , Y} - {\sum }_{i}^{n} {λ}_{k , k + i} \times {D}_{k + i , Y}}{1 - {\sum }_{i}^{n} {λ}_{k , k + i}}$$

where

$${\sum }_{i}^{n} {λ}_{k , k + i} \times {D}_{k + i , Y}$$

indicates the probability weighted sum of direct effect for each path that starts from variable k. If

$${\sum }_{i}^{n} {λ}_{k , k + i} = 1 ,$$

full mediation can be assumed and D_{k,Y} can be set to 0, as the formula would otherwise be undefined.

If the direct effect on the selected candidate output variable for all variables in the temporal order have been determined at decision block 424, the method 400B can proceed to decision block 428 in FIG. 4B, where a regression coefficient can be calculated for each variable. For example, the coefficient beta for regressing binary variable Y on Z₁, . . . , Zₙ can be calculated as:

$${β}_{kY} = log ( {D}_{ky} )$$

which estimated coefficient measures the log-odds increase in candidate output variable Y when a particular variable Zₖ changes from 0 (non-occurrence) to 1 (occurrence), accounting for only the direct effect of the variable on Y. When calculating the coefficient, only the direct effect matters because all indirect effects are blocked by having their values fixed based on the user-specified features. The regression intercept can be defined to be the log of prior odds of Y and can be calculated as:

$${β}_{0 Y} = log ( \frac{C ( Y = 1 )}{1 - C ( Y = 1 )} ) .$$

The method 400B can proceed to decision block 430, where it is determined if sufficient variables (e.g., based on user-specified features) have been considered in evaluating candidate output variable Y. For example, in observational data there can be a significant amount of confounding that can distort findings. In some embodiments, to remove confounding, the variables can be stratified, for example, such that there are “parents” in the Markov Blanket of treatment. Parents refer to large predictors of treatment that also precede it and their effect is not mediated through other variables. For example, parents can be identified through Least Absolute Shrinkage and Selection Operator (LASSO) logistic regressions, which select m largest beta coefficients in prediction of treatment (e.g., not necessarily prediction of outcome Y, but prediction of treatment Tx). These variables may be responsible for selection bias and statistically controlling them can reduce confounding in the data and improve causal interpretation of the findings.

In some cases, users may not report a complete set of parents of treatment, for example, because patients may not report symptoms or conditions they do not have. Yet, the specification of the status of these variables may be necessary for improving accuracy of the findings. Thus, variables that may be parents of a candidate output value (e.g., treatment) can be identified, and the m variables whose estimated logistic regression coefficients β_{k,Tx} have the largest absolute values (e.g., the m predictors most strongly associated (positively or negatively) with the candidate output variable) can be selected. Such an analysis can be conducted for all possible candidate output variables prior to the user interacting with the system (e.g., prior to process block 402), for example, stored as part of the predetermined knowledgebase.

In some embodiments, once the parents of the candidate output variable have been identified (e.g., through analysis of population level data), then the user can be asked (e.g., via interaction with the intake module) to verify if the parent variables are present or absent. In some embodiments, this verification may be part of the initial receipt of medical history information (e.g., as part of process block 402). Alternatively or additionally, in some cases, some of the variables needed for removing selection bias may not have been mentioned by the user in their interactions with the intake module. In such cases, the method 400B may proceed from decision block 430 to process block 432, where additional information is requested regarding the patient, for example, to verify the presence or absence of these missing variables.

In some embodiments, process block 432 can include having the medical analysis module instruct the intake module to ask the user to verify the presence or absence of the missing variables. For example, such instructions to the intake module may be:
“You are an intelligent system verifying that all necessary information has been collected about the patient's medical history. Tell the patient thank you for the information you have provided so far. Besides the information you have provided, a number of other pieces of information could affect the advice of this system. Just to clarify, you do not have history of any of the following conditions: [List the top “r” medical events that are parents of treatment].”
   Based on the responses received from the user (which, in some cases, may involve further extracting 404 and matching 406, for example, to the missing variables or other variables), the method 400B can proceed to process block 434, where the model of direct effects of variables on the selected candidate output value produced by process blocks 416-422 can be updated.

If there were no missing variables at decision block 430, or after the update of process block 434, the method 400B can proceed to process block 436, where a likelihood coefficient can be calculated for the selected candidate output value. For example, the final likelihood, L(Y|Z₁, . . . , Zₙ), of the candidate output value Y, given the patient reported medical history events, can be computed as:

$$L ( Y \vert {Z}_{1} , … , {Z}_{n} ) = \frac{1}{1 + {e}^{- {β}_{0 y} - {\sum }_{i} {β}_{iy} {Z}_{i}}} .$$

The method 400B can proceed to decision block 438, where it is determined if there are additional candidate output variables for consideration. If there remain additional candidate output variables, the method 400B can select the next candidate output variable at process block 440 and return to process block 412 in FIG. 4A to repeat the method to calculate the final likelihood for the next selected candidate output variable. Otherwise, if all candidate output variables have been considered, the method 400B can proceed to process block 442, where one or more of the candidate output variables can be output, for example, as a recommended medical treatment or medical diagnosis.

In some embodiments, the output of process block 442 may include a single candidate output variable. For example, if one treatment is better than the rest by more than 5%, then a single treatment is recommended. Alternatively or additionally, in some embodiments, the output of process block 442 may include multiple candidate outputs variables. For example, if multiple treatments are within 5% of each other, no single treatment would be recommended. Rather, the intake module can mention all of the treatments with roughly equivalent outcomes. Alternatively, in some embodiments, the output of process block 442 may include no candidate output variables. For example, if no treatment is likely to lead to positive outcome (e.g., all treatments have probability of positive outcome less than 0.10), then no treatment is recommended. In such cases, the intake module may encourage the user to look into other treatments that were not part of the knowledgebase.

In some embodiments, the output of process block 442 may include an explanation of how the candidate output variable was selected. For example, in logistic regression, the coefficients of the model can be used to list variables that explain the prediction of the regression. In a similar fashion, the estimated coefficients β_{iy} can be used to explain the findings of the algorithm. Alternatively or additionally, in some embodiments, sensitivity analysis can be performed across models constructed for different candidate output variables, for example, to see if addition or deletion of a variable can change the recommendations.

In some embodiments, the output can be from the medical analysis module to the intake module, and can include instructions to report the candidate output variables in a substantially-hallucination-free manner to the user. For example, the intake module can be instructed to provide advice using pre-drafted text or a script, without use of generative features of the intake module. In some embodiments, the intake module may change how the advice is worded, but key elements of the advice (e.g., which treatment is most effective) may be immutable. In some embodiments, the output of process block 442 may result in the user being provided with, for example, (1) a summary of the advice (e.g., one or two sentences), (2) a listing of the user-supplied information, as well as an advisory that that if this information is not correct, the advice will be misleading, and (3) a provision of the treatment recommendation or medical diagnosis, based on predicted probability calculated from prediction of Y. For example, such instructions to the intake module may be:
“You are an intelligent computer system providing advice about accuracy of diagnoses and effectiveness of treatment. Do not refer to yourself as a person but be clear that you are a computer. Let the user know that the system is ready to provide its advice. Then provide the following advice. Follow the text provided here verbatim.
Start with a summary of the recommendation. Say that the AI system recommends citalopram. Say that this medication is most likely to lead to remission of the patient's symptoms, according to experiences of more than 3 million patients with different antidepressants. Among cases with similar medical history as the patient, 45% experienced remission of their major depression symptoms. Tell the user that this rate of remission was higher than 14 other antidepressants that were examined.
Say that the system's advice could change if the information supplied is not correct. Identify which information supports and which information contradicts use of citalopram. Say that among the information provided, the following medical history supported the use of citalopram: <List variables with positive beta coefficients>.
Say the following medical history events you supplied reduced the likelihood that citalopram is effective: <list the variables with negative coefficients>.
Say that on balance, the analysis recommends that you use citalopram because the probability of remission is higher than other common medications, including: <list the medications examined>.
Say that errors are likely. Encourage the patient to consult his or her clinician, before acting on the computer's recommendation. Warn that the patient should not suddenly change or stop taking antidepressants. Sudden changes in use of antidepressants can lead to suicide. Say that the clinician can provide the final advice regarding whether they should change or start mediations. The clinician can indicate if our advice is appropriate for the patient.
Be brief. Allow the user to ask clarifying questions about side effects of citalopram and the method AI used to arrive at its conclusions. Allow the user to change the patient's medical history if what is reported here is not correct. Once the patient has finished asking clarification questions, ask if the system could follow up to see if the patient has met with their clinician and how they did.
   From the perspective of the user, the advice from the intake module seems like any AGI generated text; however, the advice is actually restricted to pre-set text, allowing no change in intention of the advice, thereby avoiding, or at least reducing, hallucinations.

Although blocks 402-442 of method 400A/400B have been described as being performed once, in some embodiments, multiple repetitions of a particular block may be employed before proceeding to the next decision block or process block. In addition, although blocks 402-442 of method 400A/400B have been separately illustrated and described, in some embodiments, process blocks may be combined and performed together (simultaneously or sequentially). Moreover, although FIGS. 4A-4B illustrate a particular order for blocks 402-442, embodiments of the disclosed subject matter are not limited thereto. Indeed, in certain embodiments, the blocks may occur in a different order than illustrated or simultaneously with other blocks. In some embodiments, method 400A/400B can include steps or other aspects not specifically illustrated in FIGS. 4A-4B. Alternatively or additionally, in some embodiments, method 400A/400B may comprise only some of blocks 402-442 of FIGS. 4A-4B.

### Computer Implementation Examples

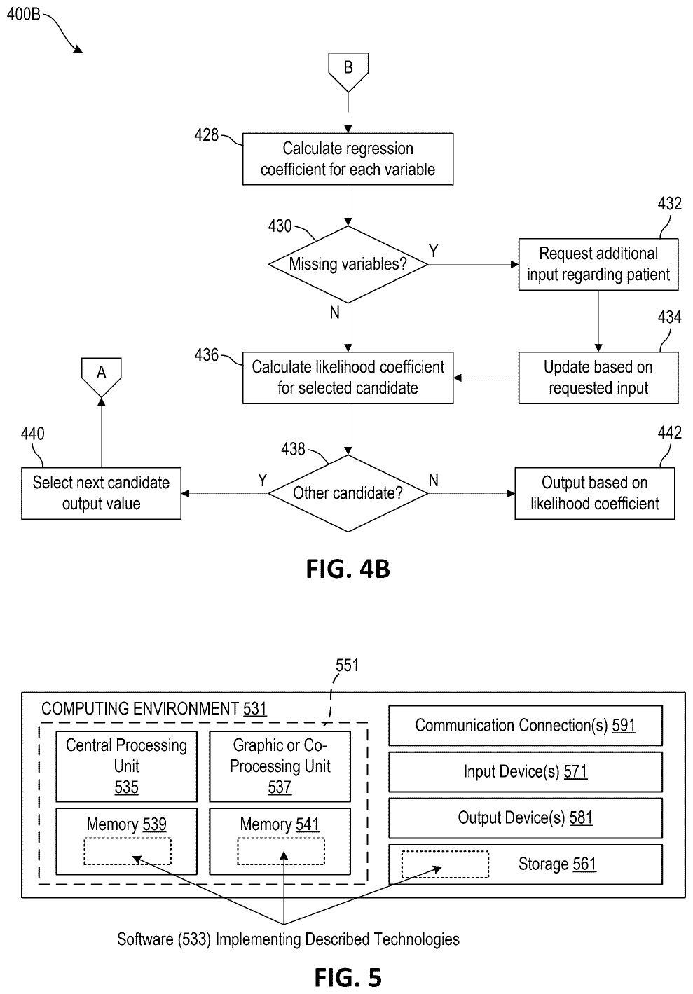

FIG. 5 depicts a generalized example of a suitable computing environment 531 in which the described innovations may be implemented, such as but not limited to aspects of medical analysis module 104, intake module 106, client device 108, supervisor device 110, MAGI system 200, and/or method 400A/400B. The computing environment 531 is not intended to suggest any limitation as to scope of use or functionality, as the innovations may be implemented in diverse general-purpose or special-purpose computing systems. For example, the computing environment 531 can be any of a variety of computing devices (e.g., desktop computer, laptop computer, server computer, tablet computer, etc.).

With reference to FIG. 5, the computing environment 531 includes one or more processing units 535, 537 and memory 539, 541. In FIG. 5, this basic configuration 551 is included within a dashed line. The processing units 535, 537 execute computer-executable instructions. A processing unit can be a central processing unit (CPU), processor in an application-specific integrated circuit (ASIC), or any other type of processor (e.g., hardware processors, graphics processing units (GPUs), virtual processors, etc.). In a multi-processing system, multiple processing units execute computer-executable instructions to increase processing power. For example, FIG. 5 shows a central processing unit 535 as well as a graphics processing unit or co-processing unit 537. The tangible memory 539, 541 may be volatile memory (e.g., registers, cache, RAM), non-volatile memory (e.g., ROM, EEPROM, flash memory, etc.), or some combination of the two, accessible by the processing unit(s). The memory 539, 541 stores software 533 implementing one or more innovations described herein, in the form of computer-executable instructions suitable for execution by the processing unit(s).

A computing system may have additional features. For example, the computing environment 531 includes storage 561, one or more input devices 571, one or more output devices 581, and one or more communication connections 591. An interconnection mechanism (not shown) such as a bus, controller, or network interconnects the components of the computing environment 531. Typically, operating system software (not shown) provides an operating environment for other software executing in the computing environment 531, and coordinates activities of the components of the computing environment 531.

The tangible storage 561 may be removable or non-removable, and includes magnetic disks, magnetic tapes or cassettes, CD-ROMs, DVDs, or any other medium which can be used to store information in a non-transitory way, and which can be accessed within the computing environment 531. The storage 561 can store instructions for the software 533 implementing one or more innovations described herein.

The input device(s) 571 may be a touch input device such as a keyboard, mouse, pen, or trackball, a voice input device, a scanning device, or another device that provides input to the computing environment 531. The output device(s) 581 may be a display, printer, speaker, CD-writer, or another device that provides output from computing environment 531.

The communication connection(s) 591 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, radio-frequency (RF), or another carrier.

Any of the disclosed methods can be implemented as computer-executable instructions stored on one or more computer-readable storage media (e.g., one or more optical media discs, volatile memory components (such as DRAM or SRAM), or non-volatile memory components (such as flash memory or hard drives)) and executed on a computer (e.g., any commercially available computer, including smart phones or other mobile devices that include computing hardware). The term computer-readable storage media does not include communication connections, such as signals and carrier waves. Any of the computer-executable instructions for implementing the disclosed techniques as well as any data created and used during implementation of the disclosed embodiments can be stored on one or more computer-readable storage media. The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or any other such network) using one or more network computers.

For clarity, only certain selected aspects of the software-based implementations are described. Other details that are well known in the art are omitted. For example, it should be understood that the disclosed technology is not limited to any specific computer language or program. For instance, aspects of the disclosed technology can be implemented by software written in C++, Java™, Python®, and/or any other suitable computer language. Likewise, the disclosed technology is not limited to any particular computer or type of hardware. Certain details of suitable computers and hardware are well known and need not be set forth in detail in this disclosure.

It should also be well understood that any functionality described herein can be performed, at least in part, by one or more hardware logic components, instead of software. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

Furthermore, any of the software-based embodiments (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, and infrared communications), electronic communications, or other such communication means. In any of the above-described examples and embodiments, provision of a request (e.g., data request), indication (e.g., data signal), instruction (e.g., control signal), or any other communication between systems, components, devices, etc. can be by generation and transmission of an appropriate electrical signal by wired or wireless connections.

### MAGI Processing Examples

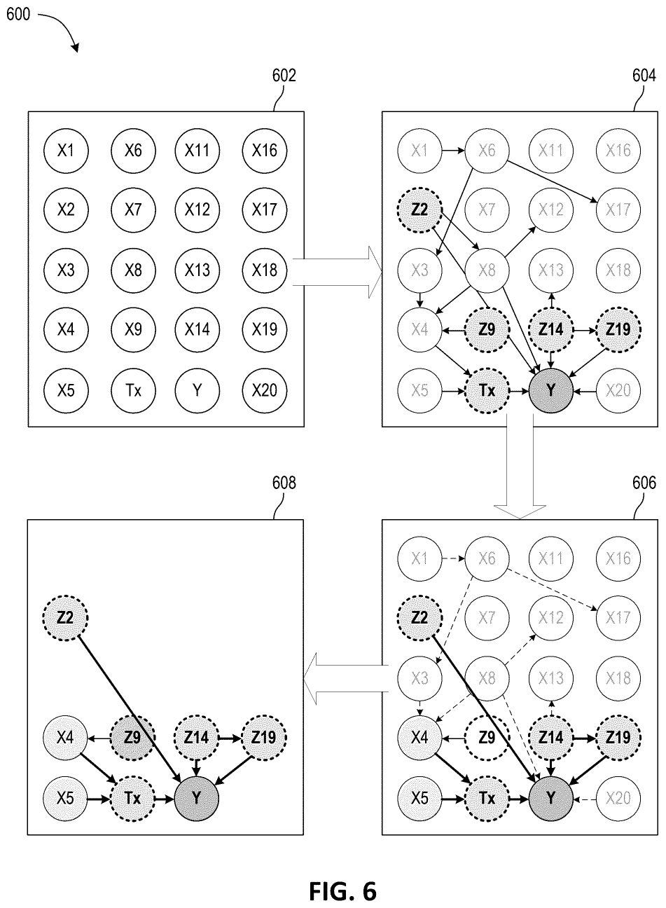

To better demonstrate certain principles of the disclosed subject matter, FIG. 6 graphically illustrates processing 600 of a knowledgebase 602 by a MAGI system based on patient-specified features and with respect to a candidate output variable. In the illustrated example, Y represents response to treatment, Xs indicate concepts in the knowledgebase of MAGI, Zs indicate features that the patient has reported through its conversation with the intake module, and Tx indicates a treatment the patient is considering. The patient has indicated the presence or occurrence of variables Z2, Z9, Z14, and Z19, represented by dashed line nodes in the updated knowledgebase 604, and the system is attempting to predict the effect of Tx on Y. In the updated knowledgebase 604, the solid-line nodes with Xs indicate variables seen in the population but not reported by the patient. Some of the reported variables map to known relationships in the knowledgebase. For example, Z2 is associated with X8 (an event not in the patient's medical history and not reported to the intake module). Moreover, Z14 is associated with Y, an event that the system is attempting to predict.

For each variable, the total impact of the variable on its subsequent variables is known from the predetermined knowledgebase, which total impact includes both direct and indirect effects through other variables. The system thus further processes the knowledgebase to estimate direct effects of the patient-specified variables on the considered treatment Y, for example, as shown via further processed knowledgebase 606. The solid link links in knowledgebase 606 indicate direct impact of a variable on another, while dashed lines represent total effects between pairs of variables.

In further processed knowledgebase 606, a direct link can be observed between Z2 and Y, which is included in the collapsed network model 608. Z14 has two paths to Y, one direct and the other indirect through Z19, both of which are included in the collapsed network model 608. In addition, Z19 has a direct path to Y, and Tx has one path to Y. The links for both are included in the collapsed network model 608. Although X4 and X5 were note reported by the patient, they can be include as parents to the treatment variable (e.g., they have a large association with treatment Tx, precede Tx, and have no intermediary node between them and Tx).

However, Z9 does not have a direct path to Y in further processed knowledgebase 606; rather, there is only an indirect path that is mediated by X4, and then goes to Tx and finally to Y. In some embodiments, this path can be ignored until X4 becomes an observed node, for example, when the patient additionally reports in response to a request for confirmation based on a sufficiency determination, as described elsewhere herein. For example, the patient can be asked to report on X4 and X5, even if initially they have not reported on these variables. When these variables are reported to be either present or absent, then the parents in the Markov blanket of treatment are blocked and thus no backdoors exist between Y and treatment. Thus, the effect of treatment is measured without confounding and the system can ignore or unlearn all other variables that indirectly affect treatment selection. All other associations/likelihoods are ignored and classified as irrelevant to the treatment selection.

The original knowledgebase 602 provides the total impact of any single variable on Y, but it does not provide the effect of the combination of the variables on Y. Embodiments of the disclosed subject matter can thus use the reduced knowledgebase 608 to combine the impact of patient reported variables, shown as variables Z1, . . . , Zn, on variable Y, to more reliably predict Y while using fewer computational resources.

To further illustrate the principles of the disclosed subject matter, computational steps associated with calculating temporal order and direct effects are described below for a simplified knowledgebase of four medical history events (e.g., Cyst procedure, Cornea thickness problem, Response to citalopram, and Anesthesia procedure), as shown in Table 1 below. Embodiments of the disclosed subject matter are not limited to specifics to the described computational steps. Indeed, practical implementations of embodiments of the disclosed subject matter would involve many more variables and computational steps, and thus would be incapable of performance outside the context of a computing environment.

For example, using the data in the knowledgebase of Table 1, the temporal order of the variables (e.g., four medical history events) can be determined. For each variable Zₖ, the score for number of events that precede can be calculated as:

$$Score ( {Z}_{k} ) = {\sum }_{j \neq k} C ( {Z}_{j} << {Z}_{k} ) - C ( ( {Z}_{k} << {Z}_{j} ) )$$

where a higher score indicates the more variables that precede Zₖ. The variables can then be sorted based on the score to produce the order of the variables. Y can be assumed to be a dependent variable and occurring after all independent variables, such that the order of variables will be Z₁<<Z₂<< . . . <<Zₙ<<Y.

TABLE 1
Example knowledgebase for four medical history events

| | | Number | | Code & | Target | | Number | Target | Code |
|---|---|---|---|---|---|---|---|---|---|
| | | of | Code & | Not | No | No | of | Before | Before |
| Target | Code | Target | Target | Target | Code | Target | Code | Code | Target |
| Response | Cyst | 11159 | 117 | 1488 | 11042 | 25153 | 1605 | 66 | 50 |
| Response | Cornea | 11159 | 11 | 86 | 11148 | 25153 | 97 | 1 | 10 |
| Response | Anesthesia | 11159 | 380 | 4756 | 10779 | 25153 | 5136 | 212 | 168 |
| Cyst | Response | 1605 | 117 | 11042 | 1488 | 34707 | 11159 | 50 | 66 |
| Cyst | Cornea | 1605 | 1 | 96 | 1604 | 34707 | 97 | 1 | 0 |
| Cyst | Anesthesia | 1605 | 54 | 5082 | 1551 | 34707 | 5136 | 34 | 20 |
| Cornea | Response | 97 | 11 | 11148 | 86 | 36215 | 11159 | 10 | 1 |
| Cornea | Cyst | 97 | 1 | 1604 | 96 | 36215 | 1605 | 0 | 1 |
| Cornea | Anesthesia | 97 | 13 | 5123 | 84 | 36215 | 5136 | 6 | 7 |
| Anesthesia | Response | 5136 | 380 | 10779 | 4756 | 31176 | 11159 | 168 | 212 |
| Anesthesia | Cyst | 5136 | 54 | 1551 | 5082 | 31176 | 1605 | 20 | 34 |
| Anesthesia | Cornea | 5136 | 13 | 84 | 5123 | 31176 | 97 | 7 | 6 |

Using the data in Table 1, the number of times that a target variable occurs before each of the other variables was calculated, and the results are shown in Table 2 below. For example, the “Anesthesia procedure” occurs 212 times before and 168 times after “Response to citalopram,”, and thus the net times the “Anesthesia procedure” occurs before “Response to citalopram” would be 168−212=−44. In Table 2, the total for each row indicates the number of events that occur before the row heading. Thus, on balance, there are 57 events where “Anesthesia procedure” occurs after the other 3 events. The variable listed first in the temporal order is the variable having most prior events. In the example of Table 2, “Response to citalopram” occurs prior to all other events 66 times, so it is listed as the first variable. For example, the initial order of occurrence based on Table 2 would be: (1) Response to citalopram, (2) Cornea thickness problem, (3) Cyst procedure, and (4) Anesthesia procedure. However, since the outcome of interest in the analysis is Response to citalopram (and by definition all outcomes are forced to occur last), the temporal order can be reset as: (1) Cornea thickness problem, (2) Cyst procedure, (3) Anesthesia procedure, and (4) Response to citalopram.

TABLE 2
Temporal order calculations for four medical history events

| Medical | | | | | |
|---|---|---|---|---|---|
| History Event | Anesthesia | Cornea | Cyst | Response | Total |
| Anesthesia | — | 1 | −14 | −44 | −57 |
| Cornea | −1 | — | −1 | 9 | 7 |
| Cyst | 14 | 1 | — | −16 | −1 |
| Response | 58 | −9 | 17 | — | 66 |

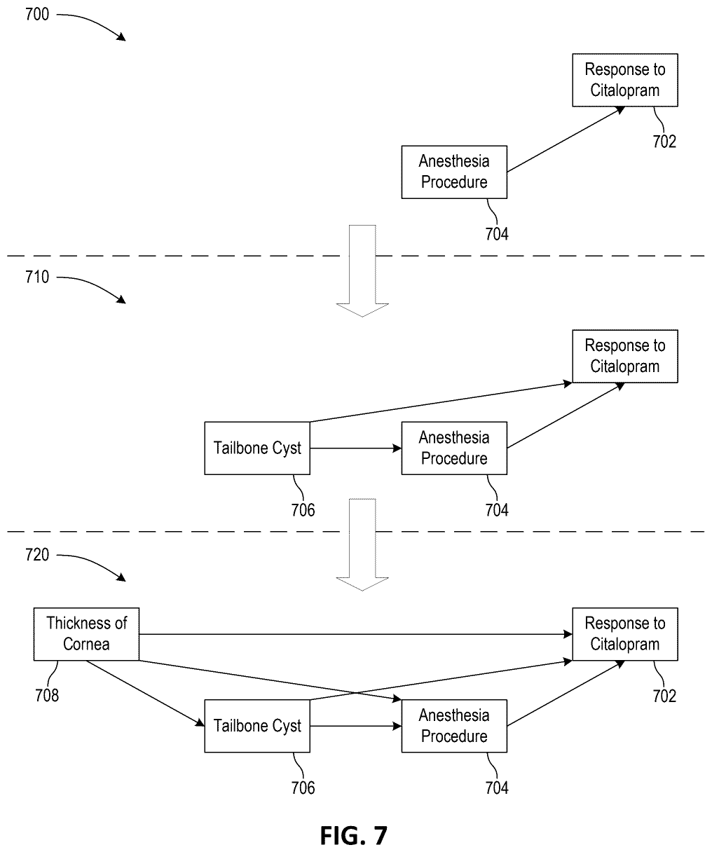

Using the data in Table 1, the direct effect of each of the variables on “Response to citalopram” can be calculated through a series of recursive steps. For example, the analysis can start with the last two events in the temporal order: “Anesthesia procedure” 704 and “Response to citalopram” 702, as shown schematically by graph 700 in FIG. 7. The total effect of “Anesthesia procedure” on “Response to citalopram” can be calculated from Table 1 as:

$${T}_{ar} = \frac{L ( Y = 1 \vert {Z}_{k} = 1 )}{L ( Y = 0 \vert {Z}_{k} = 1 )} / \frac{L ( Y = 1 \vert {Z}_{k} = 0 )}{L ( Y = 0 \vert {Z}_{k} = 0 )}$$

$${T}_{ar} = \frac{C ( Response ⋂ Anesthesia )}{C ( No Response ⋂ Anesthesia )} / \frac{C ( Response ⋂ No Anesthesia )}{C ( No Response ⋂ No Anesthesia )}$$

$${T}_{ar} = \frac{380}{4756} / \frac{10779}{20397} = 0.151$$

where index “a” indicates “Anesthesia procedure,” index “r” indicates “Response to citalopram,” and Tₐᵣ shows the odds of “Response to citalopram” given the “Anesthesia procedure.” Since there is no mediator between the last two variables, the direct effect, Dₐᵣ, can be the same as the total effect, Tₐᵣ, i.e., Dₐᵣ=Tₐᵣ=0.151

Moving recursively backwards, the last three events in the temporal order can then be selected: “Cyst procedure” 706, “Anesthesia procedure” 704, and “Response to citalopram” 702, as shown schematically by graph 710 in FIG. 7. Similar to the previous step, the total effect of “Cyst procedure” on “Response to citalopram” can be calculated from the knowledgebase in Table 1 as:

$${T}_{sr} = \frac{C ( Response ⋂ Cyst )}{C ( No Response ⋂ Cyst )} / \frac{C ( Response ⋂ No Cyst )}{C ( No Response ⋂ No Cyst )} = 0.169$$

where index “s” indicates “Cyst procedure” and Tₛᵣ reflects the total impact of “Cyst procedure” on “Response to citalopram.” The direct impact of “Cyst procedure” can then be determined by removing the mediating impact of the “Anesthesia procedure”, for example, as:

$${D}_{sr} = \frac{{T}_{sr} - {λ}_{sa} \times {D}_{ar}}{1 - {λ}_{sa}}$$

where λₛₐ is the likelihood of occurrence of “Anesthesia procedure” for patients who have “Cyst procedure” and can be calculated from the knowledgebase in Table 1 as:

$${λ}_{sa} = L ( Anesthesia = 1 \vert Cyst = 1 ) = \frac{54}{1605} = 0.034 .$$

The direct effect of “Cyst procedure” on “Response to citalopram” can thus be calculated as:

$${D}_{sr} = \frac{0.169 - 0.034 \times 0.151}{1 - 0.034} = 0.170 .$$

Moving recursively backwards, the last four events in the temporal order can then be selected: “Cornea thickness problem” 708, “Cyst procedure” 706, “Anesthesia procedure” 704, and “Response to citalopram” 702, as shown schematically by graph 720 in FIG. 7. Similar to the previous steps, the total effect of “Cornea thickness problem” on “Response to citalopram” can be calculated from the knowledgebase in Table 1 as:

$${T}_{cr} = \frac{C ( Response ⋂ Cornia )}{C ( No Response ⋂ Cornia )} / \frac{C ( Response ⋂ No Cornia )}{C ( No Response ⋂ No Cornia )} = 0.288$$

where index “c” indicates “Cornea thickness problem” and T_{cr} reflects the total impact of “Cornea thickness problem” on “Response to citalopram.” The direct impact of “Cornea thickness problem” can then be determined by removing the mediating impacts of the “Anesthesia procedure” and the “Cyst procedure,” for example, as:

$${D}_{cr} = \frac{{T}_{cr} - {λ}_{cs} \times {D}_{sr} - {λ}_{ca} \times {D}_{ar}}{1 - {λ}_{cs} - {λ}_{ca}}$$

where λ_{cs} is the likelihood of occurrence of “Cyst procedure” for patients who have “Cornea thickness problem” and λ_{ca} is the likelihood of occurrence of “Anesthesia procedure” for patients who have “Cornea thickness problem,” both of which can be calculated from the knowledgebase in Table 1 as:

$${λ}_{cs} = L ( Cyst = 1 \vert Cornia = 1 ) = \frac{1}{97} = 0 \text{.010}$$

$${λ}_{ca} = L ( Anesthesia = 1 \vert Cornia = 1 ) = \frac{13}{97} = 0.134 .$$

The direct effect of “Cornea thickness problem” on “Response to citalopram’ can thus be calculated as:

$${D}_{cr} = \frac{0.288 - 0.010 \times 0.170 - 0.134 \times 0.151}{1 - 0.010 - 0.134} = 0.311$$

The above calculations provide the direct odds ratio of the effect for occurrence of each variable on the outcome of interest.

The coefficient beta for regressing binary variable Y on Z₁, . . . , Zₙ can then be calculated as:

$${β}_{kY} = log ( {D}_{kY} )$$

$${β}_{Anesthesia , Response} = log ( 0.159 ) = - 1.8 \; 93$$

$${β}_{Anesthesia , Response} = log ( 0.17 ) = - 1.7 \; 72$$

$${β}_{Anesthesia , Response} = log ( 0.159 ) = - 1.1 \; 68$$

$${β}_{0 , Response} = log ( \frac{11159}{25153} ) = - 0.813$$

and the final likelihood for the “Response to citalopram” can be calculated for the combined effect of the 3 patient-reported binary variables anesthesia, cyst, and cornea as:

$$L ( Y \vert {Z}_{1} , … , {Z}_{n} ) = \frac{1}{1 + {e}^{- {β}_{0 y} - {\sum }_{i} {β}_{iy}}}$$

$$L ( Response \vert Anesthesia , Cyst , Cornia ) = \frac{1}{1 + {e}^{( 0.813 + 1.893 + 1.772 + 1.168 )}} = 0.004 .$$

### CONCLUSION

Any of the features illustrated or described herein, for example, with respect to FIGS. 1-7, can be combined with any other feature illustrated or described herein, for example, with respect to FIGS. 1-7 to provide systems, devices, modules, methods, and embodiments not otherwise illustrated or specifically described herein. All features described herein are independent of one another and, except where structurally impossible, can be used in combination with any other feature described herein. In view of the many possible embodiments to which the principles of the disclosed technology may be applied, it should be recognized that the illustrated embodiments are only examples and should not be taken as limiting the scope of the disclosed technology. Rather, the scope is defined by the following claims. Applicant therefore claims all that comes within the scope and spirit of these claims.

1. A method comprising:
(a) receiving, at an intake module, medical history information of a patient;
(b) extracting at least one patient-specific feature from the received medical history information, each feature corresponding to a medical history event, a medical diagnosis, a medical symptom, a medical procedure, a medication, a treatment, a response to treatment, an outcome, or a laboratory finding;
(c) correlating, via a medical analysis module, each feature to at least one of a plurality of variables in a predetermined knowledgebase, the predetermined knowledgebase including data indicative of an order of occurrence for the plurality of variables, the plurality of variables including a plurality of candidate output variables;
(d) creating, via the medical analysis module, a reduced knowledgebase from the predetermined knowledgebase based at least in part on the correlated variables;
(e) analyzing, via the medical analysis module, the reduced knowledgebase with respect to at least one of the plurality of candidate output variables; and
(f) identifying, via the medical analysis module, one or more of the candidate output variables based at least in part on the analyzing of the reduced knowledgebase,
wherein the plurality of candidate output variables comprise different medical diagnoses or different responses to medical treatments.

2. The method of claim 1, further comprising, after (f):
selecting, via the medical analysis module, a predetermined script for reporting the identified one or more candidate output variables; and
transmitting, via a hallucination-free large language model of the intake module, the selected predetermined script.

3. The method of claim 1, wherein:
the analyzing of (e) comprises identifying at least one variable, which potentially has a direct effect for the at least one of the plurality of candidate output variables, that is missing from the reduced knowledgebase; and
the method further comprises, prior to (f):
requesting, via the intake module, further information from a user regarding the at least one variable missing from the reduced knowledgebase;
receiving, via the intake module, the further information from the user; and
updating, via the medical analysis module, the reduced knowledgebase based at least in part on the received further information.

4. The method of claim 1, wherein the intake module employs one or more large language models.

5. The method of claim 1, wherein:
the receiving medical history information is via a conversation between the intake module and a user, and
the medical analysis module manages the conversation to move between topics, ask clarifying questions, and/or ask for additional information from the user.

6. The method of claim 1, wherein at least some of the plurality of variables in the predetermined knowledgebase correspond to structured codes from medical ontology.

7. The method of claim 1, wherein the creating of (d) comprises:
calculating a direct effect for each correlated variable with respect to the at least one of the plurality of candidate output variables, and
removing at least some variables from the predetermined knowledgebase that are not correlated to any extracted feature.

8. The method of claim 7, wherein the reduced knowledgebase comprises a directed acyclical graph including the correlated variables and based on the calculated direct effects.

9. The method of claim 1, further comprising, prior to (a), building the predetermined knowledgebase by receiving a plurality of medical records including structured codes and timestamps.

10. The method of claim 9, wherein:
a temporal order of occurrence of probabilities is calculated for each pair of variables based at least in part on the timestamps, a predetermined order of variables, or a percent of variation based on an alternative order of occurrence,
a conditional probability is calculated for each variable; and
directed arcs between related pairs of variables are based at least in part on the calculated conditional probabilities and the temporal order of occurrence.

11. A system comprising:
one or more processors;
one or more databases storing a predetermined knowledgebase including data indicative of an order of occurrence for a plurality of variables, the variables including a plurality of candidate output variables comprising different medical diagnoses or different responses to medical treatments; and
one or more non-transitory media storing computer-readable instructions that, when executed by the one or more processors, cause the one or more processors to perform functions of one or more modules, the one or more modules comprising a medical analysis module configured to:
receive at least one patient-specific feature corresponding to a medical history event, a medical diagnosis, a medical symptom, a medical procedure, a medication, a treatment, a response to treatment, an outcome, or a laboratory finding;
correlate the at least one feature to at least one of the variables in the predetermined knowledgebase;
create a reduced knowledgebase from the predetermined knowledgebase based at least in part on the correlated variables;
analyze the reduced knowledgebase with respect to at least one of the plurality of candidate output variables; and
identify one or more of the candidate output variables based at least in part on the analysis on the reduced knowledgebase.

12. The system of claim 11, wherein the one or more modules further comprise an intake module configured to receive medical history information from a user, each patient-specific feature being extracted from the received medical history information.

13. The system of claim 12, wherein the one or more databases further store a plurality of predetermined scripts; and the one or more non-transitory media store further computer-readable instructions that, when executed by the one or more processors, further cause the one or more processors to:
select one of the predetermined scripts for reporting the identified one or more candidate output variables, and
instruct a large language model of the intake module to transmit the selected predetermined script to the user in a hallucination-free manner.

14. The system of claim 12, wherein the medical analysis module is further configured to:
analyze the reduced knowledgebase by identifying at least one variable, which potentially has a direct effect for the at least one of the plurality of candidate output variables, that is missing from the reduced knowledgebase;
instruct the intake module to request further information from the user regarding the missing at least one variable; and
update, prior to identifying the one or more of the candidate output variables, the reduced knowledgebase based at least in part on the further information from the user.

15. The system of claim 12, wherein the intake module is configured to employ one or more large language models.

16. The system of claim 12, wherein:
the intake module is configured to converse with the user to receive the medical history information; and
the medical analysis module is configured to manage the conversation to move between topics, ask clarifying questions, and/or ask for additional information from the user.

17. The system of claim 11, wherein the medical analysis module is configured to create the reduced knowledgebase by:
calculating a direct effect for each correlated variable with respect to the at least one of the plurality of candidate output variables, and
removing at least some variables from the predetermined knowledgebase that are not correlated to any received feature.

18. The system of claim 11, wherein the one or more modules further comprise a knowledgebase creation module configured to build the predetermined knowledgebase by receiving a plurality of medical records including structured codes and timestamps.

19. The system of claim 18, wherein the medical analysis module is further configured to:
calculate a temporal order of occurrence of probabilities for each pair of variables based at least in part on the timestamps, a predetermined order of variables, or a percent of variation based on an alternative order of occurrence; and
calculate a conditional probability for each variable,
wherein directed arcs between related pairs of variables are based at least in part on the calculated conditional probabilities and the temporal order of occurrence.

20. The system of claim 11, further comprising an intake module separate from and electronically communicating with the medical analysis module, the intake module being configured to receive medical history information from a user, each feature being extracted from the received medical history information.
